(19) United States
(12) Patent Application Publication
(10) Pub. No.: US 2005/0203111 A1
(43) Pub. Date: Sep. 15, 2005

(54) Compositions and methods for preventing and treating skin and hair conditions

(75) Inventor: Nathaniel E. David, San Francisco, CA (US)

Correspondence Address: WILSON SONSINI GOODRICH & ROSATI, 650 PAGE MILL ROAD, PALO ALTO, CA, 94304-1050, US

(73) Assignee: VVII NewCo 2003, Inc., Menlo Park, CA (US)

(21) Appl. No.: 10/799,867

(22) Filed: Mar. 12, 2004

(51) Int. Cl. A61K 31/517; A61K 31/4745; A61K 31/4196

(52) U.S. Cl. 514/266.1; 514/383; 514/357; 514/396; 514/303

(57) ABSTRACT

The present invention discloses compositions and methods for the prevention and treatment of skin and hair diseases, such as, for example, alopecia, psoriasis, and keloids. In one embodiment, the present invention discloses a method for preventing and treating hair loss by applying locally to a region lacking hair a p38α MAP kinase inhibitor. The p38α MAP kinase inhibitor is preferably formulated as a gel, ointment, spray or solution that can be applied topically, transdermally, or subcutaneously to the targeted region.

### BACKGROUND OF THE INVENTION

The present invention relates to compositions and methods for preventing and treating skin and hair conditions.

The skin is the second largest organ in the body and is of primary importance to the survival of a mammal. The skin rests on subcutaneous tissue largely composed of a loose mesh of collagen fiber, fat cells, and muscle tissue. An average adult has over 3,000 square inches of skin surface area. Overall, fat-free skin accounts for at least 6 percent of an individual's total weight. The density of structures in the skin varies considerably depending on its location. But on average, one square centimeter of skin contains about 10 hair follicles, 15 sebaceous glands, 100 sweat glands, half a meter of blood vessels, 2 meters of nerves with 3,000 sensory cells at the ends of nerve fibers, 200 nerve endings to record pain, 25 pressure receptors for the perception of tactile stimuli, 2 sensory receptors for cold, and 12 sensory receptors for heat.

The skin of a mammal is derived from ectoderm and mesoderm layers of an embryo. These two layers give rise to the epidermis and dermis, respectively. The ectoderm and mesoderm layers also give rise to specialized appendages including sensory nerves, sweat glands, and hair follicles. Thus, the skin and hair follicles are physiologically related.

The skin serves various functions including, but not limited to, providing flexible physical support, maintaining constant temperature, excreting waste materials such as salts and water, producing vitamins by photochemical reactions in the skin, sensory functions, providing protection against the excesses of ultraviolet light by pigmentation such as melanin, providing protection of organs, preventing absorption of unwanted or dangerous chemicals, and providing an immunological defense.

Hair serves similar functions. The main function of hair is to provide protection against heat loss. Hair may also act to protect the epidermis from minor abrasions and from ultraviolet light. In addition, hair may provide indication of sexual development. It may also play an important role in attracting mates by indicating the general health and vitality of an individual. Furthermore, certain body parts may contain specialized hairs. Specialized hair such as eyebrows and eyelashes act to protect the eyes by channeling or sweeping away fluids, dust and debris. Nasal hair act to trap air borne foreign particles before they reach the lungs. These specialized hairs and other hair follicles have a highly developed nerve network around them that can provide sensory, tactile information about the environment.

There are many conditions that affect skin and hair. Such conditions include, but are not limited to, acne, scarring, vitiligo, and hair loss. It would be desirable to identify novel methods and compositions for preventing and/or treating skin and hair conditions.

### SUMMARY OF THE INVENTION

The present invention relates to methods for treating and/or preventing skin and hair conditions.

In particular, the present invention relates to methods for treating and/or preventing hair loss in a patient by administering to such patient an effective amount of one or more p38 inhibitors. The p38 inhibitors are preferably administered locally to a region requiring hair regeneration or prevention of hair loss. More preferably, the p38 inhibitors are administered topically, transdermally or subcutaneously.

The present invention also relates to methods for treating and/or preventing skin conditions, such as, for example, vitiligo and acne, and acne scars by administering to such a patient an effective amount of one or more p38 inhibitors. Again, the p38 inhibitors are preferably administered locally. More preferably, the p38 inhibitors are administered topically, transdermally or subcutaneously.

Examples of p38 inhibitors include, but are not limited to, pyridinylimidazoles, substituted pyrazoles, substituted pyridyls, quinazoline derivatives, aryl ureas, heteroaryl analogues, substituted imidazole compounds, and substituted triazole compounds.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates p38 inhibitor BIRB-796.

FIG. 2 illustrates p38 inhibitor CNI-1493.

FIG. 3 illustrates p38 inhibitor RDP-58.

FIG. 4 illustrates p38 inhibitor VX-745.

### INCORPORATION BY REFERENCE

All publications and patent applications mentioned in this specification are herein incorporated by reference to the same extent as if each individual publication or patent application was specifically and individually indicated to be incorporated by reference.

### DETAILED DESCRIPTION OF THE INVENTION

The present invention relates to compositions and methods for preventing and treating skin and hair conditions. The compositions of the present invention include at least one p38 MAP kinase (referred to herein as “p38”) inhibitor. The term “p38” as used herein, refers to all isoforms, splicing variants, homologues, fragments, metabolites, prodrugs, and mimetics of p38, both naturally occurring and synthetic. The term “p38 inhibitor,” as used herein, refers to any agent that blocks, diminishes, inhibits, hinders, limits, decreases, reduces, restricts or interferes with the activity of endogenous p38. A p38 inhibitor can also function upstream or downstream of p38 to downregulate the amount or function of p38.
p38 is a stress-activated protein. p38 can be activated by, for example, UV light, heat, chemical or osmotic shock, IL-1, TNF, and endotoxins. p38 is one of three families of MAP kinases: the extracellular regulated kinases (ERKs), the c-Jun NH2 terminal kinases or stress activated protein kinases (JNKs or SAP kinases), and the p38 MAP kinases. A distinguishing feature of each of these kinase families is that the ERKs have a TEY amino acid motif, the JNKs or SAP kinases have a TPY amino acid motif, and the p38 MAP kinases have a TGY amino acid motif.

The p38 family includes four different isoforms: p38α MAP kinase (p38α), p38β MAP kinase (p38β), p38γ MAP kinase (p38γ), and p38δ MAP kinase (p38δ). p38α is expressed ubiquitously. A shorter C-terminal truncated form of p38α known as Mxi-2 has also been identified in a yeast two-hybrid screen based on its association with the transcription factor Max. p38β has been shown to have an additional isoform, p38β2 that lacks the 8 amino acid insertion found in p38β. Between these two variants p38β2 is believed to be the major form as p38β is catalytically less active. p38γ and p38δ are 63% and 61% identical to p38α, respectively. p38γ is expressed predominantly in skeletal muscle wherein p38δ is expressed predominantly in testes pancreas, prostate, small intestine, and endocrine tissue.

All p38 homologues and splice variants contain a 12 amino acid activation loop between kinase domain VII and kinase domain VIII. The activation loop includes a Thr-Gly-Tyr motif. Dual phosphorylation of both Thr-180 and Tyr-182 (p38α numbering) in the TGY motif is essential for the activation of p38 resulting in >1000 fold increase in specific activity of these enzymes. Dual phosphorylation can be effected by MKK6, MKK3 and other members of the MAPKK (mitogen activating protein kinase kinase) family and MAPKKK (mitogen activating protein kinase kinase kinase) family, also referred to as the MAP3K family. In particular, MEKK4/MTK1, ASK1, and TAK1 have been identified as upstream activators of MAP3K. Also, TNF-stimulated activation of p38α is believed to be mediated via recruitment of TRAF2 (TNF receptor associated factor) and the Fas adaptor protein, Dazz, which results in the activation of ASK1 and subsequently p38 and JNK. Also, TAK has been shown to activate MKK6 in response to TGF-β and is believed to be associated with TRAF6 in an IL-1-dependent manner suggesting involvement of TAK1 in IL-1-mediated p38 activation. Additionally, mixed lineage of kinase-3 physically associated with MKK3 and MKK6 is believed to be involved in activation of p38 by Ste-20-linked kinases. Also, MEKK3, small G proteins of the Rho family, and active forms of Cdc42 and Racl in mammalian cells have also been shown to activate the p38 pathways (the latter via p21-activation kinase).

Thus, p38 is a key control point in the cellular immune system. In particular, p38 exerts its effects by regulating the production of cytokines. p38 is activated by phosphorylation on Thr-180 and Tyr-182 by MEKs (MKK3 or MKK6), and in response to that, p38 phosphorylates MAPJAP2 kinase, which relieves post-transcriptional repression of TNF-α and IL1 transcripts by phosphorylating (and thus inactivating) a AU-rich binding protein that binds to the 3′-UTR of the TNF and IL1. mRNAs. Because multiple stress pathways are able to activate p38, p38 inhibition is able to broadly suppress cytokine (e.g., TNF-α, IFN-α, IL1) production and its resulting activation of the immune system.

To date, there have been several mechanisms and numerous compounds suggested for the inhibition of p38. Compounds that have been suggested for the inhibition of p38 include pyridinylimidazoles. See Young P. R., et al., (1997) J. Biol. Chem. 272, 12116-12121; see also Bender, P. E., (1985) J. Med. Chem. 28, 1169-1177. Examples of pyridinylimidazoles that may inhibit p38 include 6-(4′-fluorophenyl)-5-(4′-pyridyl)-2,3-dihydroimidazo(2,1-b)-thia zole and its metabolites (sulfoxide, sulfone), analogues, fragments, and mimetics. It has further been suggested that the minimal structure of pyridinylimidazoles, 4-(pyridin-4-yl)-5-phenylimidazole, may be sufficient to inhibit p38. See Gallagher, T F, et al., (1997) Bio-org. Med. Chem. 5, 49-64.

Certain 1,5-diaryl-substituted pyrazole compounds have also been suggested as p38 inhibitors. Such substituted pyrazole compounds are disclosed in U.S. Pat. No. 6,509,361, assigned to Pharmacia Corporation, incorporated herein by reference for all intended purposes. Additional pyrazole derivatives that inhibit p38 are disclosed in U.S. Pat. No. 6,335,336, assigned to G.D. Searle & Co., incorporated herein by reference for all intended purposes.

Other p38 inhibitors include substituted pyridyl, such as those disclosed in U.S. Patent Application Publication No. 2003/0139462, incorporated herein by reference for all intended purposes.

Additional p38 inhibitors are those disclosed in U.S. Pat. No. 6,610,688, assigned to Sugen, Inc., incorporated herein by reference for all intended purposes.

Quinazoline derivatives may also function as p38 inhibitor. Examples of quinazoline derivatives that are p38 inhibitors are disclosed in U.S. Pat. Nos. 6,541,477 and 6,184,226, assigned to Scios Inc., incorporated herein by reference for all intended purposes, and U.S. Pat. Nos. 6,509,363 and 6,635,644, assigned to Vertex Pharmaceuticals Inc., incorporated herein by reference for all intended purposes.

Aryl ureas and heteroaryl analogues may also function as p38 inhibitors. Examples of aryl ureas and heteroaryl analogues that are p38 inhibitors are disclosed in U.S. Pat. No. 6,344,476, assigned to Bayer Corp., incorporated herein by reference for all intended purposes. WO99/32110, published Jul. 1, 1999, describes heterocyclic ureas as p38 kinase inhibitors. WO99/32463, published Jul. 1, 1999, describes urea compounds that inhibit p38 kinase. WO98/52558, published Nov. 26, 1998, describes urea compounds for the inhibition of p38 kinase. WO99/00357, published Jan. 7, 1999, describes the use of urea compounds as inhibitors of p38 kinase. WO99/58502, published Nov. 18, 1999, describes urea compounds as inhibitors of p38 kinase. These and all other references mentioned herein are incorporated by references for all purposes.

Substituted imidazole compounds and substituted triazole compounds may also function as p38 inhibitors. Such compounds are disclosed in U.S. Pat. Nos. 6,560,871 and 6,599,910, respectively, which incorporated herein by reference for all intended purposes.

Additional p38 inhibitors include RWJ-67657 (RW Johnson Pharmaceutical Research Institute); RDP-58 (SangStat Medical Corp.); RDP-58; Scios-323 (Scios Inc.); Scios-469 (Scios Inc.); MKK3/MKK6 inhibitors (Signal Research Division); p38/MEK modulators (Signal Research Division); SB-210313 analogs, SB-220025, SB-238039, HEP-689, SB-203580, SB-239063, SB-239065, SB-242235 (SmithKline Beecham Pharmaceuticals); VX-702 and VX-745 (Vertex Pharmaceuticals Inc.); AMG-548 (Amgen Inc.); Astex p38 kinase inhibitors (Astex Technology Ltd.); RPR-200765 analogs (Aventis SA); Bayer p38 kinase inhibitors (Bayer Corp.); BIRB-796 (Boehringer Ingelheim Pharmaceuticals Inc.); Celltech p38 MAP kinase inhibitor (Celltech Group plc.); FR-167653 (Fujisawa Pharmaceutical Co. Ltd.); 681323 and SB-281832 (GlaxoSmithKline plc); LEO Pharmaceuticals MAP kinase inhibitors (LEO Pharma A/S); Merck & Co. p38 MAP kinase inhibitors (Merck research Laboratories); SC-040 and SC-XX906 (Monsanto Co.); Novartis adenosine A3 antagonists (Novartis AG); p38 MAP kinase inhibitors (Novartis Pharma AG); CP-64131 (Pfizer Inc.); CNI-1493 (Picower Institute for Medical Research); RPR-200765A (Rhone-Poulenc Rorer Ltd.); and Roche p38 MAP kinase inhibitors and Ro-320-1195 (Roche Bioscience).

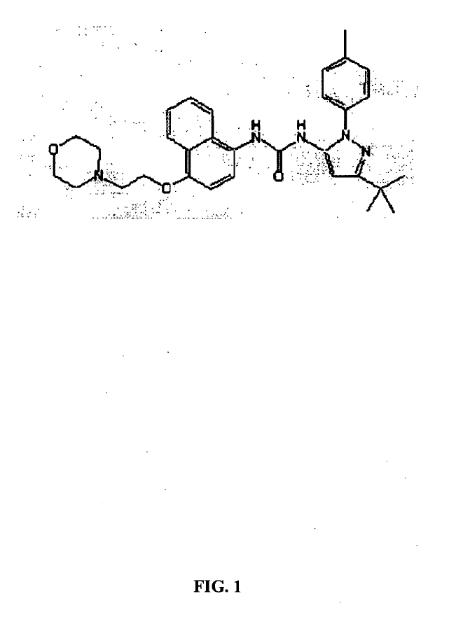

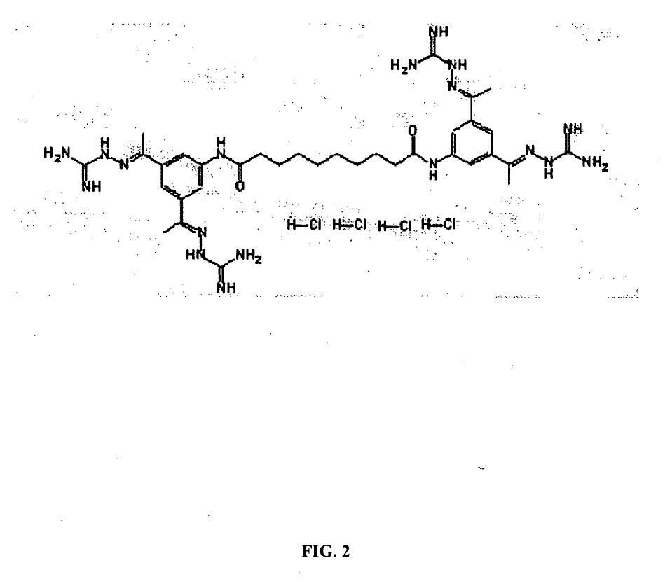

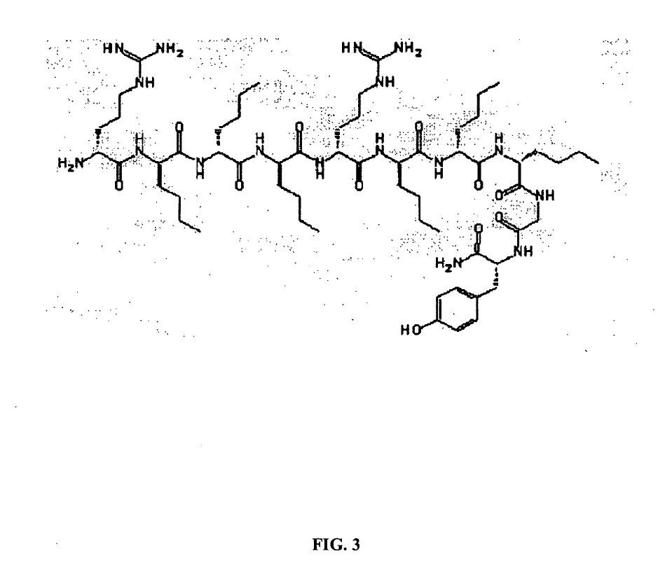

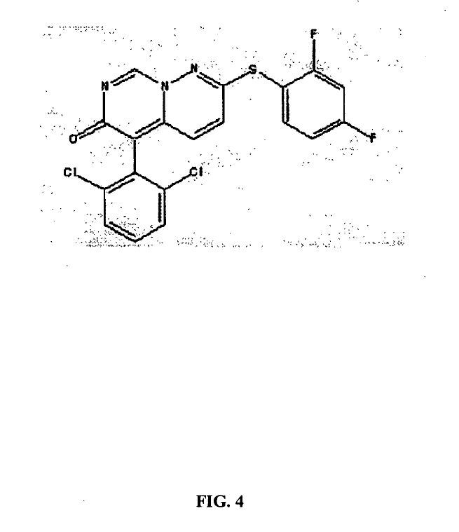

In preferred embodiments, the p38 inhibitor is RDP-58 (SangStat Medical Corp.), AMG-548 (Amgen Inc.), BIRB-796 (Boehringer Ingelheim Pharma.), CNI-1493 (Picower Institue for Medical Research), VX-702 or VX-745 (Vertex Pharmaceuticals Inc.). FIG. 1 illustrates p38 inhibitor BIRB-796. FIG. 2 illustrates p38 inhibitor CNI-1493. FIG. 3 illustrates p38 inhibitor RDP-58. FIG. 4 illustrates p38 inhibitor VX-745.

The present invention also relates to compositions that include at least one p38 inhibitor, and that may optionally include one or more additional active agents. Active agents can include, for example, anti-inflammatory agents, immunomodulators, antibacterial agents, antiviral agents, and/or antifungal agents.

Anti-inflammatory agents include, but are not limited to, pyrazolones, fenamate, diflunisal, acetic acid derivatives, propionic acid derivatives, oxicams, mefenamic acid, Ponstel™, meclofenamate, Meclomen™, phenylbutazone, Butazolidin™, diflunisal, Dolobid™, diclofenac, Voltaren™, indomethacin, Indocin™, sulindac, Clinoril™, etodolac, Lodine™, ketorolac, Toradol™, nabumetone, Relafen™, tolmetin, Tolectin™, ibuprofen, Motrin™, fenoprofen, Nalfon™, flurbiprofen, Ansaid™, carprofen, Rimadyl™, ketoprofen, OrudiS™, naproxen, Anaprox™, Naprosyn™, piroxicam, and Feldene™.

The term “immunomodulator” as used herein includes cytokines, stem cell growth factors, lymphotoxins, co-stimulatory molecules, hematopoietic factors, and synthetic analogs of these molecules. Examples of immunomodulators include tumor necrosis factor, interleukins (e.g., interleukin-1 (IL-1), IL-2, IL-3, IL-4, IL-5, IL-6, IL-7, IL-8, IL-9, IL-10, IL-11, IL-12, IL-13, IL-14, and IL-15), colony stimulating factors (e.g., granulocyte-colony stimulating factor and granulocyte macrophage-colony stimulating factor), interferons (e.g., interferons-α,β,γ,δ,ε,Ω,T), the stem cell growth factor designated “S1 factor,” erythropoietin, and thrombopoietin. Additional examples of immunomodulators include, but are not limited to, azathioprine (Imuran), 6-mercaptopurine (6-MP, Purinethol), cyclosporine (Sandimmune), and methotrexate.

Examples of antibacterial agents include, but are not limited to, a tetracycline, a sulfa drug, a penicillin, a quinolone, a cephalosporin, and mixtures thereof. Exemplary tetracyclines include doxycycline and minocycline. An exemplary sulfa drug includes sulfacetamde. An exemplary cephalosporin includes cephalexin (commercially available as KEFLEX). Exemplary quinolones include the floxacins, such as loemfloxacin, ofloxacin, and ciprofloxacin.

Examples of antiviral agents include, but are not limited to, acyclovir, tamvir, penciclovir, and the like, and mixtures thereof.

Examples of anti-fungal agents include but are not limited to, famesol, econazole, fluconazole, clotrimazole, ketoconazole, calcium or zinc undecylenate, undecylenic acid, butenafine hydrochloride, ciclopirox olainine, miconazole nitrate, nystatin, sulconazole, terbinafine hydrochloride, and the like, and mixtures thereof.

It should be readily understood that any salts, isomers, prodrugs, metabolites, or other derivatives of these anti-microbial agents may also be included as the anti-microbial agent in accordance with the invention.

A pharmaceutical composition of the present invention may be formulated to be suitable for application in a variety of manners, for example, in a cream for topical application to the skin (e.g., for alopecia), in a wash, in a douche, in a powder for chaffing (e.g., for dermatitis), in a liquid, in a dry formulation (e.g., as a bath salt or bath powder), and the like. Other formulations will be readily apparent to one skilled in the art. In preferred embodiments, the compositions herein are preferably formulated for local administration. Preferably, the compositions are formulated for topical, subcutaneous or transdermal administration.

When formulated as an ointment, the active ingredient (e.g., a p38 inhibitor) can be employed, for example, with either paraffinic or a water miscible ointment base. Alternatively, the active ingredients can be formulated in a cream with an oil-in-water cream base. If desired, the aqueous phase of the cream base can include, for example at least 30% w/w of a polyhydric alcohol such as propylene glycol, butane-1,3-diol, mannitol, sorbitol, glycerol, polyethylene glycol and mixtures thereof.

The topical formulations can desirably include a compound that enhances absorption or penetration of the active ingredient through the skin or other affected areas. Examples of such dermal penetration enhancers include dimethylsulfoxide and related analogs.

The pharmaceutical compositions herein may also include, for example, antioxidants (e.g., vitamin E); buffering agents; lubricants (e.g., synthetic or natural beeswax); sunscreens (e.g., para-aminobenzoic acid); and other cosmetic agents (e.g., coloring agents, fragrances, oils, essential oils, moisturizers or drying agents). Thickening agents (e.g., polyvinylpyrrolidone, polyethylene glycol or carboxymethylcellulose) may also be added to the compositions.

The carriers utilized in the pharmaceutical compositions of the present invention may be solid-based dry materials for use in powdered formulations or may be liquid or gel-based materials for use in liquid or gel formulations. The specific formulations depend, in part, upon the routes or modes of administration.

Typical carriers for dry formulations (e.g., bath salts) include, but are not limited to, trehalose, malto-dextrin, rice flour, micro-crystalline cellulose (MCC), magnesium sterate, inositol, fructo-oligosaccharides FOS, gluco-oligosaccharides (GOS), dextrose, sucrose, talc, and the like carriers. Where the composition is dry and includes evaporated oils that produce a tendency for the composition to cake (i.e., adherence of the component spores, salts, powders and oils), it is preferable to include dry fillers which both distribute the components and prevent caking. Exemplary anti-caking agents include MCC, talc, diatomaceous earth, amorphous silica and the like, typically added in an concentration of from approximately 1% to 95% by-weight.

Suitable liquid or gel-based carriers are well-known in the art (e.g., water, physiological salt solutions, urea, methanol, ethanol, propanol, butanol, ethylene glycol and propylene glycol, and the like). Preferably, water-based carriers are approximately neutral pH.

Suitable carriers include aqueous and oleaginous carries such as, for example, white petrolatum, isopropyl myristate, lanolin or lanolin alcohols, mineral oil, fragrant or essential oil, nasturtium extract oil, sorbitan mono-oleate, propylene glycol, cetylstearyl alcohol (together or in various combinations), hydroxypropyl cellulose (MW=100,000 to 1,000,000), detergents (e.g., polyoxyl stearate or sodium lauryl sulfate) and mixed with water to form a lotion, gel, cream or semi-solid composition. Other suitable carriers comprise water-in-oil or oil-in-water emulsions and mixtures of emulsifiers and emollients with solvents such as sucrose stearate, sucrose cocoate, sucrose distearate, mineral oil, propylene glycol, 2-ethyl-1,3-hexanediol, polyoxypropylene-15-stearyl ether and water. For example, emulsions containing water, glycerol stearate, glycerin, mineral oil, synthetic spermaceti, cetyl alcohol, butylparaben, propylparaben and methylparaben are commercially available. Preservatives may also be included in the carrier including methylparaben, propylparaben, benzyl alcohol and ethylene diamine tetraacetate salts. Well-known flavorings and/or colorants may also be included in the carrier. The composition may also include a plasticizer such as glycerol or polyethylene glycol (MW 400 to 20,000). The composition of the carrier can be varied so long as it does not interfere significantly with the pharmacological activity of the active ingredient (p38 inhibitor).

The compositions and pharmaceutical compositions herein may be used to prevent and treat skin and hair conditions.

Examples of skin conditions include, but are not limited to, acne, acne scars, scleroderma, psoriasis, atopic dermatitis, vitiligo, keloid, hypertrophic scars, and vascularity. Skin conditions also include any condition that causes irritation, inflammation, infection or discoloration of skin.

The term acne refers to plugged pores (blackheads and whiteheads), pimples, papules, pustules, macules, cysts or nodules. Acne can occur on all body parts and can affect people of all ages. While not life threatening, acne can often lead to scarring which may be permanent.

Acne may result from hair follicle blockage. The hair follicle blockage allows for sebum (oil), which normally drains to the surface of the skin, to aggregate and for bacteria to grow. It is postulated that androgen may be involved in causing acne, and that the sebaceous glands of people with acne react differently, or excessively, to normal levels of androgen hormones. Normally, skin cells in the follicles grow, mature, die, flake off and are carried to the surface of the skin by the flow of sebum. However, for acne patients, it is suggested that dead cells fail to be carried to the surface and instead block the inside of the follicle, trapping oil and bacteria (e.g., P. acnes), which in turn lead to acne.

When the trapped sebum and bacteria stay below the skin surface, a whitehead is formed. On the other hand, when the trapped sebum and bacteria open to the surface they turn black due to melanin, the skin's pigment, and a blackhead is formed. Blackheads can last for a long time because the contents very slowly drain to the surface.

Whiteheads and blackheads are also referred to as comedo. A comedo is a sebaceous follicle plugged with sebum, dead cells from inside the sebaceous follicle, tiny hairs, and sometimes bacteria. Neither blackheads nor whiteheads should be squeezed or opened, unless it is done under sterile conditions. This is to prevent subsequent skin infection by bacteria (e.g., staphylococci).

In more severe forms of the disease papules, pastures, nodules, and cysts may be formed. A papule is a small, solid lesion slightly elevated above the surface of the skin. A papule is usually less than 5 mm across. A papule is believed to be caused by localized cellular reaction to the process of acne. A group of papules and microcomedones (blackheads and whiteheads) may be almost invisible but can create a bumpy appearance to the skin.

Like a papule, a nodule is a solid, dome-shaped or irregularly-shaped lesion. However, unlike a papule, a nodule is characterized by inflammation that extends into deeper layers of the skin and may cause tissue destruction and/or scarring. A nodule may be very painful. Nodular acne is a severe form of acne that may not respond to therapies other than isotretinoin.

A pustule is a dome-shaped lesion that contains pus. The pus usually consists of a mixture of white blood cells, dead skin cells, and bacteria. It is common for a pustule that forms over a sebaceous follicle to have a hair in its center. Acne pustules that heal without progressing to cystic form usually do not leave scars.

A macule is the temporary red spot left by a healed acne lesion. A macule is generally flat, red or red-pink, and has a well defined border. A macule may persist for days or weeks before disappearing. When a number of macules are present at one time they can contribute to the “inflamed face” appearance of acne.

A cyst is a sac-like lesion containing liquid or semi-liquid material. The liquid often consists of white blood cells, dead cells, and bacteria. A cyst is larger than a pustule and may be severely inflamed down into deeper layers of the skin. Like nodules, a cyst may be very painful and may result in scarring.

Cysts and nodules often occur together in a severe form of acne called nodulocystic. Systemic therapy with isotretinoin is sometimes the only effective treatment for nodulocystic acne.

Thus, the present invention involves administering to a patient suffering from or susceptible to acne an effective amount of one or more of the compositions herein. Such compositions include at least one p38 inhibitor. Such compositions are preferably administered locally (e.g., topically, transdermally, or subcutaneously). Any composition herein can be administered independently or in combination with one or more additional agents or treatments. Such agents and/or treatments include, but are not limited to, retinoids, antibiotics, oral contraceptives, Accutane, laser treatment (e.g., Smoothbeam), isotretinoin, etc. The p38 inhibitor may be administered prior to, simultaneous with, or after the administration of additional agents and/or treatments. Preferably, the p38 inhibitor will be administered prior to the administration of the additional agent (e.g., a retinoid, an antibiotic or isotretinoin).

The present invention also contemplates the prevention and/treatment of acne scars. Scars, also known medically as cicatrix, are marks left by a healed wound, burn, or incision, and are composed of tough fibrous tissue. There are many forms of scars, including but not limited to, acne scars, keloids, hypertropic scars, pigmentary scars, hormone induced scars, animal bite scars, etc.

Acne scars are a unique form of scars that can occur anywhere on the body. Acne scars can be of various shapes, sizes, and depth. It is thought that acne scars are caused by the activation of the immune system in fighting acne bacteria. Generally, humans and other mammals recognize invading microorganisms by recognizing their microbial patterns, e.g., (1) LPS-lipopolysaccharide, mannose, fucose, and other sugar residues, (2) techoid acid, or (3) N-formyl peptides. These, and other, microbial patterns are recognized by pattern recognition molecules (PRMs) or pattern recognition receptors (PRRs). Examples of PRR's include, f-Met-Leu-Phe receptors, which bind to N-formyl peptides and attract neutrophils; complement receptors (CRs), which bind to complement components such as C3b and C4b; macrophage mannose receptors, which bind to mannose residues commonly present on surface of microorganisms; scavenger receptors, which recognize certain anionic polymers and acetylated low-density lipoproteins; and CD 14 receptors on the surface of phagocytes, which allow for the recognition of LPS.

It is postulated that acne bacteria activates the innate immune system via activation of the f-Met-Leu-Phe receptors. Activation of the innate immune system by f-Met-Leu-Phe receptors activates p38, which has also been shown to be present in scar formation. Thus, by inhibiting p38, it may be possible to reduce scarring that results from acne or other effects resulting from the activation of the innate immune system. Activation of p38 can be temporary or long term (even permanent).

Current treatments for acne scars include, but are not limited to, dermabrasion, laser resurfacing, chemical peels, punch techniques, subcision, and augmentation. Dermabrasion involves removal of damaged skin using a quickly rotating diamond edged wheel or other abrasive device. Depending on how coarse the wheel or the device is, one can control the amount of skin that is removed. Laser resurfacing involves the use of a laser to remove skin so new skin can form in its place. Common lasers used include the CO₂ laser and the erbium (YAG) laser. Chemical peels involve the application of different types of acid to the skin in order to remove the top layer so that a smoother layer can surface. Punch techniques include: punch replacement, punch excision, and punch elevation. Punch replacement involves the removal of pitted scar with a hair-transplant type punch, which is then replaced with a skin graft, usually from behind the ear. This is usually the most successful method for removal of deep scars. Punch excision involves the removal of a pitted scar. The wound is then closed and allowed to heal. Finally, punch elevation involves cutting the scar loose from the bottom, but not discarding it. The scar is thus allowed to float up to the level of surrounding skin. Subcision involves detaching a scar from deeper tissue, which allows a pool of blood to form under the scar. The blood clot then helps form connective tissue under the scar, leveling it with the surface. Furthermore, augmentation involves injecting material, such as collagen and/or fat, under the scar to bring it to surface level (may follow subcision).

Thus, the present invention relates to the prevention and/or treatment of scars, or more preferably acne scars, or more preferably acne scars caused by acne cysts or nodules. In preferred embodiments, a p38 inhibitor is administered locally, such as topically, subdermally, or subcutaneously. The p38 inhibitor can be administered independently or in combination with one or more additional agents or treatments. Such agents and/or treatments include, but are not limited to dermabrasion, laser resurfacing, chemical peels, punch techniques, subcision, and augmentation. For example, a p38 inhibitor can be administered prior to, simultaneous with, or after a dermabrasion treatment, laser treatment, chemical peel, punch treatment, subcision and/or augmentation. The amount and frequency of administering the p38 inhibitor and/or additional treatments will depend on various factors (e.g., age of patient, location of acne scar, number of treatment cycles, skin coloration, etc.).

Another example of a skin condition or scar that can be treated by the present invention is a keloid. A keloid is an overgrowth of dense fibrous scar tissue that usually develops after healing at a sight of skin injury. Keloid formation is associated with excessive amounts of collagen, overproduction of which is a skin cell response to injury. A keloid typically grows beyond the boundaries of the original wound, but it rarely extends into the underlying subcutaneous tissue. Keloids are typically is raised and nodular. Keloids can range in their consistency from soft and doughy to rubbery hard. Early keloid lesions are often erythematous. The lesions are first brownish red and later become pale. Lesions are usually devoid of hair follicles and other functioning adnexal glands.

Once a keloid region occurs, its clinical course may vary. Most keloids continue to grow for weeks or months and others for years. Growth is usually slow, but keloids may occasionally enlarge rapidly, tripling in size within months. Once a keloid stops growing, it is usually asymptomatic and remains stable.

Keloids have a high recurrence rate, with over 50% of excised keloids recurring within several years after excision. While keloids are generally a cosmetic concern, they can sometimes cause contractures which may result in a loss of function if they are located over a joint or on the face.

Keloids are more common in people with dark skin complexions. For example, it is estimated that keloids form more frequently in Polynesians and Chinese than in Indians and Malaysians. Moreover, it is estimated that as many as 16% of black Africans have keloids. Whites and albinos are the least affected by keloids. Keloids are also more common in young women than in young males. However, it is believed that this abnormality is related to the fact that more your women pierce their ears than men, causing a physical injury to the skin that may result in a keloid. Keloids occur at a higher rate in individuals aged 10-30 years. Keloids occur less frequently at the extremes of ages, although an increasing number of presternal keloids have resulted from coronary artery bypass operations and other similar procedures now undertaken in older patients.

It is believed that keloid formation is linked to a genetic component and, therefore, keloid formation tends to run in families. Keloids are thought to be associated genetically with human leukocyte antigen B14, human leukocyte antigen B21, human leukocyte antigen Bwl6, human leukocyte antigen Bw35, human leukocyte antigen DR5, human leukocyte antigen DQw3, and blood group A. Transmission is reported as both autosomal dominant and autosomal recessive.

A hypertrophic scar is somewhat similar to a keloid. Like a keloid, it is associated with excessive amounts of collagen overproduction that results from a skin cell's response to injury. However, unlike a keloid, it remains within the boundaries of the original trauma or injury and is typically flat and smooth. Hypertrophic scars have a tendency for spontaneous regression over time.

Treatment of keloids and hypertrophic scars depends upon their location, size, depth, age of the patient, and past response to treatment. Currently treatments include the use of occlusive dressings, compression therapy, intralesional corticosteroid injections, cryosurgery, excision, radiation therapy, laser therapy, interferon therapy, and imiquimod 5% cream (see Berman, B., eMedicine Journal, September 6 (2001) Vol. 2, No. 9, at http://www/arabmedmag.com/issue-31-05-2003/dermatology/main05.htm).

Thus, the present invention involves the prevention and treatment of scars (e.g., keloids and hypertrophic scars) using one or more of the compositions herein containing a p38 inhibitor. A composition containing a p38 inhibitor may be administered independently or in combination with one or more additional agents and/or treatments. Examples of agents and/or treatments that may be useful in a combination treatment include, but are not limited to, occlusive dressings, compression therapy, intralesional corticosteroid injections, cryosurgery, excision, radiation therapy, laser therapy, interferon therapy, and imiquimod. The composition containing the p38 inhibitor is preferably administered locally, e.g., topically, transdermally, or subcutaneously. The p38 inhibitor may be administered prior to, simultaneous with, or after the administration of an additional agent. Preferably, the p38 inhibitor is administered prior to the administration of an additional agent or treatment.

Another common skin and hair disease is scleroderma. Scleroderma is believed to be an autoimmune disease that involves the gradual hardening and tightening of the skin due to excessive collagen production. This results in the “suffocation” of hair follicles, which in turn atrophy. The excess collagen production occurs in patches, which results in hair loss that occurs in distinct areas.

While scleroderma may develop spontaneously, it is believed that it may be induced in people who work with silica, vinyl-chloride, after silicone implants or after injection of certain drugs. Bone marrow transplant recipients and people who contract hepatitis C are also believed to be more likely to develop scleroderma. Scleroderma is three times more common in women than in men. Furthermore, it is believed that at least some of those affected by scleroderma are genetically susceptible to the condition.

The first symptoms of scleroderma often involve a premature graying of the hair followed by hair loss. When hair loss occurs on the scalp, treatment can include surgery to remove the affected skin region.

There are several different classifications of scleroderma that are distinguishable based on the progressive stage of the disease. “Localized scleroderma” refers to a small region of skin affected by scleroderma. Localized scleroderma may often be associated with a patchy hair loss. “CREST,” or “calcinosis (calcium deposits in soft tissue), Raynaud's phenomenon (hypersensitivity of the digits to cold), esophageal involvement (difficulty swallowing), sclerodactyly (skin hardening on fingers), and telangiectasis (dilation of blood vessels around the mouth),” is a more progressive form of scleroderma. While fairly benign, CREST may result in an occasional heart failure. Progressive systemic sclerosis (PSS) is the most progressive form of the disease. PSS is the result of a continued fibrosis in any or all these organs. In PSS, the scleroderma affects internal as well as external parts of the body. For example, joints, gut, lungs, kidneys, nerves and muscles (including those of the heart) may be affected by PSS.

It is believed that the overproduction of collagen which results in scteroderma results from lymphocyte cells that produce cytokines which in turn stimulate fibroblast cells and promote collagen production. In the heart, collagen overproduction and fibrosis can lead to rhythm disturbances and heart failure.

Psoriasis is another skin disease that affects up to 2% of the world's population. Psoriasis is a chronic, immune-mediated, non-contagious disease. It is believed that psoriasis has a genetic component, as Caucasians are the more susceptible to this condition than other ethnic groups. While psoriasis may develop at any age, the most common age for it to begin is in the mid thirties. While the exact cause of psoriasis is still unknown, it has been shown that onset may be preceded by streptococcal infection or stress in some cases.

Clinically, psoriasis often looks like a pink patch of raised skin that is covered in small scales of flaky, white, dead skin. Psoriasis can cause itching and burning sensations. It is believed that in addition to affecting the skin, psoriasis may also causes hair loss. For example, a psoriasis plaque (affected patches of skin) may contain hair follicles that have been forced into the telogen resting stage by the condition. This results in few visible hairs being present in the psoriasis plaques. Thus, telogen effluvium is a typical form of hair loss that affects psoriasis patients. Additionally, psoriasis may sometimes cause a scarring alopecia. While the psoriasis-induced telogen effluvium is fully reversible with proper treatment, the psoriasis-induced scarring alopecia is a permanent form of hair loss. Overall, it is believed that psoriasis is caused by the immune system sending faulty signals which result in a hasten growth cycle in skin cells.

While there are no current cures for psoriasis, some treatments may be useful to control the disease. For example, a tar shampoo may treat a mild case of psoriasis, while a shampoo containing dithranol may be used to treat a more extensive form of the disease. For severe cases, a corticosteroid treatment may be helpful. A corticosteroid treatment can involve topical creams or sometimes local corticosteroid injections into the affected skin area. Recently, preparations containing calcipotroil have been shown to be very useful in treating scalp psoriasis.

Eczema is a chronic skin rash that is extremely itchy. It consists of numerous bumps (papules) or blisters that appear on inflamed, scaly skin. The papules progress into tiny blisters. Scratching of the blisters is often provoked by severe itching and may result in bleeding, ulceration and secondary infections of the affected skin.

Atopic dermatitis is a type of eczema sometimes referred to as infantile eczema or allergic eczema. Atopic dermatitis affects 10% to 12% of all children with symptoms typically appearing within the first few months of a child's life, or before the age 5. Onset of atopic dermatitis after the age of 30 is less common and is often due to exposure of the skin to harsh or wet conditions. Atopic dermatitis often occurs on both sides of the body symmetrically. Atopic dermitits can cause the skin to become inflamed with redness, swelling, cracking, weeping, crusting and scaling.

Thus, the present invention involves the prevention and treatment of scleroderma, psoriasis, eczema, and atopic dermatitis by administering locally any of the compositions herein. In particular, the present invention contemplates the administration of at least one p38 inhibitor to an affected area topically, transdermally, or subcutaneously. The composition can be administered independently and/or in combination with one or more additional agents or treatments. Examples of agents and/or treatments include, but are not limited to tar, dithranol, a corticosteroid, calcipotroil, and imiquimod.

Vitiligo is another example of a skin condition. Vitiligo results from loss of pigment which produces white patches. Any part of the body may be affected. Usually both sides of the body are affected. Common areas of involvement are the face, lips, hands, arms, legs, and genital areas. Vitiligo affects one or two of every 100 people. About half of those who develop vitiligo, develop the disease before the age of 20. About one-fifth of those who develop vitiligo have a family member with the same condition.

It is believed that vitiligo may be an autoimmune process whereby the body makes antibodies again its own melanocyte pigment cells. Melanocytes make melanin, the pigment that determines color of skin, hair, and eyes. If these cells die or cannot form melanin, the skin becomes lighter or completely white. However, most people with vitiligo are in good general health, although vitiligo may occur with other autoimmune diseases such as thyroid disease.

The degree of pigment loss in vitiligo patients can vary within each vitiligo patch. There may be different shades of pigment in a patch, or a border of darker skin may circle an area of light skin. Vitiligo often begins with a rapid loss of pigment. This may continue until, for unknown reasons, the process stops. Cycles of pigment loss, followed by times where the pigment doesn't change, may continue indefinitely. It is rare for skin pigment in vitiligo patients to return on its own. Some people who believe they no longer have vitiligo actually have lost all their pigment and no longer have patches of contrasting skin color. Although their skin is all one color, they still have vitiligo.

The course and severity of pigment loss differ with each person. Light-skinned people usually notice the contrast between areas of vitiligo and suntanned skin in the summer. Year round, vitiligo is more obvious on people with darker skin. Individuals with severe cases can lose pigment all over the body. There is no way to predict how much pigment an individual will lose. Topical corticosteroids creams containing corticosteroid compounds can be effective in returning pigment to small areas of vitiligo.

PUVA is a form of repigmentation therapy where a type of medication known as psoralen is used. This chemical makes the skin very sensitive to light. Then the skin is treated with a special type of ultraviolet light call UVA. Sometimes, when vitiligo is limited to a few small areas, psoralens can be applied to the vitiligo areas before UVA treatments. Other treatment options include a new topical class of drugs called immunomodulators.

The present invention contemplates a method for preventing or treating vitiligo by administering to a patient susceptible to or suffering from vitiligo an effective amount of a composition that includes at least one p38 inhibitor. The composition is preferably administered locally to a region affected by vitiligo or susceptible to vitil calcipotroil igo. The composition can be administered independently or in combination with one or more other agents. Other agents include, for example, corticosteroids, psoralen, immunomodulators, etc. In some embodiments, the p38 inhibitor will be administered prior to, simultaneous with, or after the administration of an additional agent. Preferably, the p38 inhibitor will be administered prior to the administration of the additional agent (e.g., a corticosteroid or psoralen).

While hair loss itself may not pose a serious health concern, it plays an important social role. Fullness of hair is often associated by society as a manifestation of youthfulness and physical condition. Thus, hair loss may impair an individual's attraction and mating ability.

Furthermore, hair on the scalp provides protection. Primarily, it protects the head from mechanical shock, heat loss, and exposure to ultraviolet (UV) light. Similarly, specialized hairs, such as eyelashes and eyebrows protect the eyes from airborne particles and sun exposure. Moreover, hair in the ear canal and nasal passages helps to filter out particles and pathogens in protecting internal organs.

The loss of hair is often a clinical manifestation of hair disease. Hair loss occurs when the number of hairs lost exceeds the number of hairs regenerated. The average human scalp is covered by approximately 100,000 hair follicles. A hair follicle is a tube-like opening in the epidermis where the hair shaft develops and into which the sebaceous glands open. Normally, roughly 50-100 hairs randomly fall out a day. This is unnoticeable because the lost hair is replaced by as new hairs daily, as each hair follicle undergoes a hair cycle.

Hair goes through a characteristic cycle consisting of an immature phase, a growing phase called anagen, a transitional phase between the growing phase and the resting phase called catagen, and finally a resting phase called telogen in which the hair stops growing awaiting to fall out. At any given time, 85 to 90% of hairs on our body are in anagen phase or growing phase, which lasts anywhere from two to five years. This phase is followed by a short regression phase, or catagen, which lasts 2-3 weeks. Approximately 1% of hair follicles are in catagen. Approximately 10-15% of hair follicles are in the resting phase, the telogen, which lasts about 3-5 months. A hair follicle typically goes through 10-20 asynchronous cycles during its lifetime. Persistent loss of more than 100 hairs, more preferably more than 150 hairs a day, more preferably more than 200 hairs a day, more preferably more than 300 hairs a day, or more preferably more than 400 hairs a day would consist a state of hair loss, or alopecia, albeit it could be temporary.

Hair conditions that leads to hair loss is often mediated by the immune system and is often associated with inflammation of the hair follicle. Examples of hair diseases that are mediated by the immune system include, but are not limited to, alopecia greata, alopecia cicatrisata, alopecia totalis, alopecia universalis, alopecia keratosis pilaris, alopecia triangularis, anagen effluvium, androgeneic alopecia, androgenetic alopecia, area celsi, bacterial follicultiis, black piedra, blackdot ringworm, cemical alopecia, cicatrical alopecia, chronic telogen effluvium, dermatophyte infection, diet deficiency induced alopecia, diffuse alopecia, dissecting cellulites, drug induced alopecia, eosinophilic pustular folliculitis, erosive pustular dermatosis, familial focal alopecia, feldman syndrome, female alopecia, female pattern baldness, follicular degeneration syndrome, folliculitis barbae, folliculitis decalvans, folliculitis keloidalis, graham-little syndrome, herpes simplex folliculitis, herpes zoster folliculitis, hot comb alopecia, involutional alopecia, ischemic alopecia, keratosis follicularis spinulosa decalvans cum ophiasi, lichen planopilaris, lipedematous alopecia, loose anagen syndrome, loose hair syndrome, male pattern baldness, mechanically induced alopecia, mixed inflammatory alopecia, non-scarring alopecia, occipital alopecia, occipital alopecia greata, ofuji syndrome, papular atrichia, pattern baldness, perifolliculitis capitis abscedens et suffodiens of hoffinan, perinevoid alopecia greata, postpartum alopecia, pseudofolliculitis barbae, pseudopelade of brocq, ringworm, sarcoidosis, scarring alopecia, telogen effluvium, thermal alopecia, tick bite induced alopecia, tinea capitis, traction alopecia, traction folliculitis, traumatic alopecia, triangular alopecia, trichomycosis axillaries, trichotillomania, tufted hair folliculitis, and vaccination induced alopecia.

Alopecia is a condition of excessive, premature hair loss. Alopecia may be caused by many factors. These facts include, but are not limited to, genetic factors, agin, or local or systemic disease.

In alopecia greata, a patient experiences a sudden loss of hair in circumscribed areas. Such patients have no obvious skin disorder or systemic disease. Any hairy area may be involved in alopecia greata. The scalp and beard are most commonly affected by alopecia greata. In some cases, such as alopecia universalis, all body hair is lost.

In female alopecia, a female patient experiences a loss of hair. Female alopecia is usually the result of genetic factors. Female alopecia is thought to be associated with hormones and an increase in male testosterone hormone. Hormone changes that occur as a result of childbirth, contraceptive pills, anemia, and menopause, for example, can cause female alopecia. It is thought that female alopecia is caused by a dominant gene that must be present in both parents which is passed down to a daughter.

Current treatments for hair loss include, but are not limited to, Minoxidil (e.g., 5% conc.), laser phototherapy, Revivogen, Toppe™, and Shen Min™. Minoxidil is a hair growth product that specifically works on the hair follicles, which have miniaturized due to male or female pattern baldness. Minoxidil forces the hair follicles to go into the growth phase. Although Minoxidil is a vasodilator its effects are not contributed to its ability to increase circulation and its exact mode of action remains unknown. Laser phototherapy is a new treatment that is believed to stimulate hair growth. Laser phototherapy can be applied using, for example, the LaserComb™. Another form of hair growth treatment is Revivogen™. Revivogen™ is a recently approved drug that blocks the enzyme, 5-alpha-reductase. Typically, 5-alpha-reductase enzyme helps generate a hormone known as dihydro-testosterone (DHT). DHT is associated with the loss of functioning in the hair follicle. Toppek™, another new system to prevent hair loss, functions by opening the hair shaft and allowing the infusion of keratin protein into the hair shaft. Furthermore, Shen Min™, a 100% natural hair nutrient, which is derived from the eastern wild rose He Shou Wu is also thought to help generate new hair growth and restore hair color. Thus, any of the above treatments (or any other known treatments) can be used in combination with the compositions herein.

Thus, the present invention relates to methods of preventing and/or treating hair loss or hair conditions, e.g., alopecia arearta and female alopecia. In particular, the present invention contemplates a method for preventing and/or treating a hair condition in a patient by administering to such a patient an effective amount of at least one p38 inhibitor. The administration of such compound is preferably made locally, e.g., topically, subcutaneously, transdermally. The administration of the p38 inhibitor(s) can be accompanied with one or more other agents (e.g., Minozidil or Revivogen) or treatments (e.g., laser photo therapy). The p38 inhibitor can be administered prior to, simultaneous with, or after the administration of the additional agent. In preferred embodiments, the p38 inhibitor is administered prior to the administration of other agents.

Topical applications to the skin or a mucous membrane using a cream, lotion, gel, oil, ointment, suspension, aerosol spray, powder, semi-solid formulation (e.g., a suppository), or article of manufacture, all formulated so as to contain a therapeutic composition of the present invention using methods well-known in the art.

As used herein an “effective amount” refers to the amount of a composition, which produces a desired outcome. For example, an “effective amount” for a therapeutic use is an amount of a composition comprising an active compound (e.g., a p38 inhibitor) that is required to provide a clinically significant increase in preventing or treating a conditions, e.g., stimulating and/or augmenting hair growth, reducing and/or eliminating vitiligo patches, etc.

The present invention also contemplates combination therapies (e.g., treatments using two or more p38 inhibitors or a combination of a p38 inhibitor and another agent). In cases of combination therapy, a synergistic effect may result such that the effect achieved with the combination of methods and compositions of this invention is greater than the sum of their effective amounts independently. Thus, the present invention contemplates a synergistic effect may occur when adminstering two or more p38 inhibitors or when administering a p38 inhibitor and another agent.

In some embodiments, the compositions herein are administered in about one to 100 applications, preferably about one to 50 applications, more preferably about one to 25 applications, or more preferably about one to 10 applications.

Each application of the compositions herein generally consists of about 1 mg to 100 g concentration of a p38 inhibitor per application, more preferably about 10 mg to 10 g concentration of a p38 inhibitor per application, or more preferably about 50 mg to 1 g concentration of a p38 inhibitor per application. In some embodiments, a daily dose consists of about 0.01 mg/kg body weight to 100 mg/kg body weight, preferably between about 0.1 mg/kg body weight and about 50 mg/kg body weight, and more preferably between about 0.5 mg/kg body weight to 30 mg/kg body weight.

Application(s) are preferably administered for a period of about one day and up to about one year. However, longer or lifelong treatments are also contemplated, especially for preventative treatments. In preferred embodiments, applications are administered about once every twelve hours and up to about once every month. Preferably, two to four applications of the therapeutic composition are administered per month, or more preferably two to four application of the therapeutic composition are administered per week, or more preferably two to four application of the therapeutic compositions are administered per day.

For topical applications, the compositions herein are preferably applied to targeted area daily, bi-weekly, weekly, or at other regular intervals. The specific route, dosage, and timing of the administration will depend, in part, on factors, including but not limited to, the age, weight, sex, and medical condition. Topical formulations can be applied as a topical gel, spray, ointment or cream containing the active ingredients (including a p38 inhibitor) in a total amount of, for example, 0.075 to 90% w/w, preferably 0.2 to 50% w/with, and most preferably 0.4 to 25% w/w.

A transdermal device can also be used to administer the compositions of the present invention. Preferably, topical administration is accomplished using a patch either of the reservoir and porous membrane type or of a solid matrix variety. In either case, the active agent is delivered continuously from the reservoir or microcapsules through a membrane into the active agent permeable adhesive, which is in contact with the skin or mucosa of the recipient. If the active agent is absorbed through the skin, a controlled and predetermined flow of the active agent is administered to the recipient. In the case of microcapsules, the encapsulating agent can also function as the membrane. The transdermal patch can include the compound in a suitable solvent system with an adhesive system, such as an acrylic emulsion, and a polyester patch.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating or preventing hair loss in a patient comprising administering to said patient an effective amount of a p38 inhibitor.

2. The method of claim 1 wherein said p38 inhibitor is selected from the group consisting of: pyridinylimidazoles, substituted pyrazoles, substituted pyridyls, quinazoline derivatives, aryl ureas, heteroaryl analogues, substituted imidazole compounds, and substituted triazole compounds.

3. The method of claim 1 wherein said p38 inhibitor is selected from the group consisting of RWJ-67657, RDP-58, RDP-58, Scios-323, Scios-469, MKK3/MKK6 inhibitors (Signal Research Division); p38/MEK modulators (Signal Research Division); SB-210313 analogs, SB-220025, SB-238039, HEP-689, SB-203580, SB-239063, SB-239065, SB-242235, VX-702, VX-745, AMG-548, Astex p38 kinase inhibitors, RPR-200765 analogs, Bayer p38 kinase inhibitors, BIRB-796, Celltech p38 MAP kinase inhibitor, 681323, SB-281832, LEO Pharmaceuticals MAP kinase inhibitors, Merck & Co. p38 MAP kinase inhibitors, SC-040, SC-XX906, Novartis adenosine A3 antagonists, p38 MAP kinase inhibitors (Novartis Pharma AG), CP-64131, CNI-1493, RPR-200765A, Roche p38 MAP kinase inhibitors, and Ro-320-1195.

4. The method of claim 3 wherein said p38 inhibitor is selected from the group consisting of RDP-58, AMG-548, BIRB-796, CNI-1493, VX-702 and VX-745.

5. The method of claim 1 wherein said p38 inhibitor is administered locally.

6. The method of claim 1 wherein said p38 inhibitor is administered topically, subcutaneously, or transdermally.

7. A method for treating or preventing a hair condition in a patient comprising administering to said patient an effective amount of a p38 inhibitor.

8. The method of claim 6 wherein said hair condition is selected from the group consisting of alopecia greata, alopecia cicatrisata, alopecia totalis, alopecia universalis, alopecia keratosis pilaris, alopecia triangularis, anagen effluvium, androgeneic alopecia, androgenetic alopecia, area celsi, bacterial follicultiis, black piedra, blackdot ringworm, cemical alopecia, cicatrical alopecia, chronic telogen effluvium, dermatophyte infection, diet deficiency induced alopecia, diffuse alopecia, dissecting cellulites, drug induced alopecia, eosinophilic pustular folliculitis, erosive pustular dermatosis, familial focal alopecia, feldman syndrome, female alopecia, female pattern baldness, follicular degeneration syndrome, folliculitis barbae, folliculitis decalvans, folliculitis keloidalis, graham-little syndrome, herpes simplex folliculitis, herpes zoster folliculitis, hot comb alopecia, involutional alopecia, ischemic alopecia, keratosis follicularis spinulosa decalvans cum ophiasi, lichen planopilaris, lipedematous alopecia, loose anagen syndrome, loose hair syndrome, male pattern baldness, mechanically induced alopecia, mixed inflammatory alopecia, occipital alopecia, occipital alopecia greata, ofuji syndrome, papular atrichia, pattern baldness, perifolliculitis capitis abscedens et suffodiens of hoffinan, perinevoid alopecia greata, postpartum alopecia, pseudofolliculitis barbae, pseudopelade of brocq, ringworm, sarcoidosis, scarring alopecia, telogen effluvium, thermal alopecia, tick bite induced alopecia, tinea capitis, traction alopecia, traction folliculitis, traumatic alopecia, triangular alopecia, trichomycosis axillaries, trichotillomania, tufted hair folliculitis, and vaccination induced alopecia.

9. The method of claim 6 wherein said p38 inhibitor is selected from the group consisting of: pyridinylimidazoles, substituted pyrazoles, substituted pyridyls, quinazoline derivatives, aryl ureas, heteroaryl analogues, substituted imidazole compounds, and substituted triazole compounds.

10. The method of claim 7 wherein said p38 inhibitor is selected from the group consisting of RWJ-67657, RDP-58, RDP-58, Scios-323, Scios-469, MKK3/MKK6 inhibitors (Signal Research Division); p38/MEK modulators (Signal Research Division); SB-210313 analogs, SB-220025, SB-238039, HEP-689, SB-203580, SB-239063, SB-239065, SB-242235, VX-702, VX-745, AMG-548, Astex p38 kinase inhibitors, RPR-200765 analogs, Bayer p38 kinase inhibitors, BIRB-796, Celltech p38 MAP kinase inhibitor, 681323, SB-281832, LEO Pharmaceuticals MAP kinase inhibitors, Merck & Co. p38 MAP kinase inhibitors, SC-040, SC-XX906, Novartis adenosine A3 antagonists, p38 MAP kinase inhibitors (Novartis Pharma AG), CP-64131, CNI-1493, RPR-200765A, Roche p38 MAP kinase inhibitors, and Ro-320-1195.

11. The method of claim 10 wherein the p38 inhibitor is selected from the group consisting of RDP-58, AMG-548, BIRB-796, CNI-1493, VX-702 and VX-745.

12. The method of claim 7 wherein said p38 inhibitor is administered locally.

13. The method of claim 7 wherein said p38 inhibitor is administered topically, subcutaneously, or transdermally.

14. The method of claim 8 wherein the condition is alopecia greata or female alopecia.

15. A method for treating or preventing vitiligo in a patient comprising administering to said patient an effective amount of a p38 inhibitor.

16. The method of claim 15 wherein the p38 inhibitor is selected from the group consisting of: pyridinylimidazoles, substituted pyrazoles, substituted pyridyls, quinazoline derivatives, aryl ureas, heteroaryl analogues, substituted imidazole compounds, and substituted triazole compounds.

17. The method of claim 15 wherein the p38 inhibitor is selected from the group consisting of RDP-58, AMG-548, BIRB-796, CNI-1493, VX-702 and VX-745.

18. The method of claim 15 wherein said p38 inhibitor is administered locally.

19. The method of claim 15 wherein said p38 inhibitor is administered topically, subcutaneously, or transdermally.

20. The method of claim 15 further comprising administering to said patient a corticosteroid, psoralen, or an immunomodulator.

21. A method for treating or preventing acne scars in a patient comprising administering to said patient a p38 inhibitor.

22. The method of claim 21 wherein the p38 inhibitor is selected from the group consisting of: pyridinylimidazoles, substituted pyrazoles, substituted pyridyls, quinazoline derivatives, aryl ureas, heteroaryl analogues, substituted imidazole compounds, and substituted triazole compounds.

23. The method of claim 21 wherein said p38 inhibitor is selected from the group consisting of RWJ-67657, RDP-58, RDP-58, Scios-323, Scios-469, MKK3/MKK6 inhibitors (Signal Research Division); p38/MEK modulators (Signal Research Division); SB-210313 analogs, SB-220025, SB-238039, HEP-689, SB-203580, SB-239063, SB-239065, SB-242235, VX-702, VX-745, AMG-548, Astex p38 kinase inhibitors, RPR-200765 analogs, Bayer p38 kinase inhibitors, BIRB-796, Celltech p38 MAP kinase inhibitor, 681323, SB-281832, LEO Pharmaceuticals MAP kinase inhibitors, Merck & Co. p38 MAP kinase inhibitors, SC-040, SC-XX906, Novartis adenosine A3 antagonists, p38 MAP kinase inhibitors (Novartis Pharma AG), CP-64131, CNI-1493, RPR-200765A, Roche p38 MAP kinase inhibitors, and Ro-320-1195.

24. The method of claim 23 wherein the p38 inhibitor is selected from the group consisting of RDP-58, AMG-548, BIRB-796, CNI-1493, VX-702 and VX-745.

25. The method of claim 21 wherein said p38 inhibitor is administered locally.

26. The method of claim 21 wherein said p38 inhibitor is administered topically, subcutaneously, or transdermally.

27. The method of claim 21 further comprising administering to said patient a treatment selected from the group consisting of dermabrasion, laser resurfacing, chemical peels, punch techniques, subcision, and augmentation.

28. The method of claim 27 wherein said p38 inhibitor is administered locally prior to said treatment.

29. A method for treating or preventing acne in a patient comprising administering to said patient an effective amount of a p38 inhibitor.

30. The method of claim 29 wherein the p38 inhibitor is selected from the group consisting of: pyridinylimidazoles, substituted pyrazoles, substituted pyridyls, quinazoline derivatives, aryl ureas, heteroaryl analogues, substituted imidazole compounds, and substituted triazole compounds.

31. The method of claim 29 wherein said p38 inhibitor is selected from the group consisting of RWJ-67657, RDP-58, RDP-58, Scios-323, Scios-469, MKK3/MKK6 inhibitors (Signal Research Division); p38/MEK modulators (Signal Research Division); SB-210313 analogs, SB-220025, SB-238039, HEP-689, SB-203580, SB-239063, SB-239065, SB-242235, VX-702, VX-745, AMG-548, Astex p38 kinase inhibitors, RPR-200765 analogs, Bayer p38 kinase inhibitors, BIRB-796, Celltech p38 MAP kinase inhibitor, 681323, SB-281832, LEO Pharmaceuticals MAP kinase inhibitors, Merck & Co. p38 MAP kinase inhibitors, SC-040, SC-XX906, Novartis adenosine A3 antagonists, p38 MAP kinase inhibitors (Novartis Pharma AG), CP-64131, CNI-1493, RPR-200765A, Roche p38 MAP kinase inhibitors, and Ro-320-1195.

32. The method of claim 31 wherein the p38 inhibitor is selected from the group consisting of RDP-58, AMG-548, BIRB-796, CNI-1493, VX-702 and VX-745.

33. The method of claim 29 wherein said p38 inhibitor is administered locally.

34. The method of claim 29 wherein said p38 inhibitor is administered topically, subcutaneously, or transdermally.

35. The method of claim 29 further comprising administering to said patient a treatment selected from the group consisting of a retinoid, an antibiotic, an oral contraceptive, Accutane, and a laser treatment.

36. The method of claim 29 wherein said p38 inhibitor is administered prior to said treatment.

37. The method of claim 1 further comprising administering to said patient an agent selected from the group consisting of Minoxidil, laser photo therapy, Revivogen, Toppe™, and Shen Min.™

38. The method of claim 7 further comprising administering to said patient an agent selected from the group consisting of Minoxidil, laser photo therapy, Revivogen, Toppe™, and Shen Min.™

39. A method for treating a skin or hair condition associated with the activation of the innate immune system comprising administering topically to affected area an effective amount of a p38 inhibitor.

40. The method of claim 39 wherein the p38 inhibitor is selected from the group consisting of RWJ-67657, RDP-58, RDP-58, Scios-323, Scios-469, MKK3/MKK6 inhibitors (Signal Research Division); p38/MEK modulators (Signal Research Division); SB-210313 analogs, SB-220025, SB-238039, HEP-689, SB-203580, SB-239063, SB-239065, SB-242235, VX-702, VX-745, AMG-548, Astex p38 kinase inhibitors, RPR-200765 analogs, Bayer p38 kinase inhibitors, BIRB-796, Celltech p38 MAP kinase inhibitor, 681323, SB-281832, LEO Pharmaceuticals MAP kinase inhibitors, Merck & Co. p38 MAP kinase inhibitors, SC-040, SC-XX906, Novartis adenosine A3 antagonists, p38 MAP kinase inhibitors (Novartis Pharma AG), CP-64131, CNI-1493, RPR-200765A, Roche p38 MAP kinase inhibitors, and Ro-320-1195.
